(19) United States
(12) Patent Application Publication
(10) Pub. No.: US 2024/0407697 A1
(43) Pub. Date: Dec. 12, 2024

(54) Implantable Medical Device for Sensing Physiological Signals

(71) Applicant: BIOTRONIK SE & Co. KG, Berlin (DE)

(72) Inventors: Jan Romberg, Berlin (DE); Marcel Starke, Eichwalde (DE); Alexander Kraus, Berlin (DE); Ravi Kiran Kondama Reddy, Portland, OR (US)

(73) Assignee: BIOTRONIK SE & Co. KG, Berlin (DE)

(21) Appl. No.: 18/702,014

(22) PCT Filed: Oct. 20, 2022
(86) PCT No.: PCT/EP2022/079220

§ 371 Date: Apr. 17, 2024

Related U.S. Application Data
(60) Provisional application No. 63/271,901, filed on Oct. 26, 2021.

(30) Foreign Application Priority Data
Nov. 9, 2021 (EP) 21207257.3

(51) Int. Cl. A61B 5/287; A61B 5/00; A61B 5/276

(52) U.S. Cl. CPC A61B 5/287; A61B 5/276; A61B 5/7221

(57) ABSTRACT

An implantable medical device for sensing physiological signals comprises an arrangement of at least a first electrode pole, a second electrode pole and a third electrode pole, said arrangement of at least the first, second and third electrode poles being configured to sense physiological signals. The implantable medical device further comprises a processing module for processing signals received via said arrangement of at least the first, second and third electrode poles. The processing module is configured to monitor cardiac activity based on a first signal received by a first pair of electrode poles of the arrangement of at least the first, second and third electrode poles and to assess a consistency of said first signal based on a second signal received by a second pair of electrode poles of the arrangement of at least the first, second and third electrode poles different then said first pair.

### CROSS-REFERENCE TO RELATED APPLICATIONS

This application is the United States National Phase under 35 U.S.C. § 371 of PCT International Patent Application No. PCT/EP2022/079220, filed on Oct. 20, 2022, which claims the benefit of European Patent Application No. 21207257.3, filed on Nov. 9, 2021, and U.S. Provisional Patent Application No. 63/271,901, filed on Oct. 26, 2021, the disclosures of which are hereby incorporated by reference herein in their entireties.

### TECHNICAL FIELD

The instant invention generally relates to an implantable medical device for sensing physiological signals which is to be implanted into a patient for carrying out a diagnostic and/or therapeutic function.

### BACKGROUND

An implantable medical device of this kind may, for example, be a pacemaker, an implantable cardioverter defibrillator, a sensor device such as a bio-sensor, or a recording device such as a loop recorder. The implantable medical device herein is configured to sense physiological signals, for example electrocardiogram signals. For example, the implantable medical device may be a recording device which is configured to record electrocardiogram signals and to communicate recorded electrocardiogram signals or information derived from recorded electrocardiogram signals to an external device in the context of a home monitoring system.

An implantable medical device as, for example, described in EP 3 278 836 B1 may, for example, comprise a housing and an arrangement of electrode poles arranged on the housing. The electrode poles herein are arranged on the housing of the implantable medical device such that the electrode poles are aligned along a longitudinal axis along which the implantable medical device extends. The electrode poles may, for example, be formed by housing segments which are made from an electrically conductive material such as a metal material and are exposed to the outside such that they may be brought into electrical contact with surrounding tissue in order to establish an electrical coupling to the tissue in an implanted state of the implantable medical device.

An implantable medical device as used in a home monitoring system shall allow for a reliable monitoring of a physiological state of a patient. In particular, using the implantable medical device it shall be possible to reliably detect an abnormal cardiac state based on recorded electrocardiogram signals or other physiological signals. If an abnormality is detected in physiological signals, the implantable medical device shall be enabled to communicate with, for example, an external device of a home monitoring system in order to trigger a message to a service center to alert medical personnel of a potential need for attention.

It herein is desirable, however, to discern a potential false alert from a true abnormality in a physiological cardiac state. For example, in case of a mechanical or electrical failure of the implantable medical device or in case of a deterioration of the electrical coupling of the arrangement of electrode poles to surrounding tissue a change in a received signal may be observed, which however is not due to a change in a physiological state, but is caused by other factors.

The present disclosure is directed toward overcoming one or more of the above-mentioned problems, though not necessarily limited to embodiments that do.

### SUMMARY

It is an object of the instant invention to provide an implantable medical device and a method for operating an implantable medical device which in a reliable way allow to sense and process physiological signals, to detect a potential abnormality in the physiological signals. Physiological signals may be electrophysiological signals, in particular electrocardiogram signals.

At least the above object is achieved by means of an implantable medical device comprising the features of claim 1.

In one aspect, an implantable medical device for sensing physiological signals, for example electrocardiogram signals, comprises an arrangement of at least a first electrode pole, a second electrode pole and a third electrode pole, said arrangement of at least the first electrode pole, the second electrode pole and the third electrode pole being configured to sense physiological signals. The implantable medical device further comprises a processing module for processing signals received via said arrangement of at least the first electrode pole, the second electrode pole and the third electrode pole. Herein, the processing module is configured to monitor cardiac activity based on a first signal received by a first pair of electrode poles of the arrangement of at least the first electrode pole, the second electrode pole and the third electrode pole and to assess a consistency of said first signal based on a second signal received by a second pair of electrode poles of the arrangement of at least the first electrode pole, the second electrode pole and the third electrode pole different than said first pair.

The implantable medical device, accordingly, comprises multiple electrode poles, which are arranged at different axial positions on the implantable medical device. “Multiple” in this context means “at least three”. By means of the different electrode poles an electrical coupling to surrounding tissue is established when the implantable medical device is implanted in a patient, such that physiological signals may be sensed using the different electrode poles.

Electrocardiogram signals or other physiological signals herein are sensed using a first pair of electrode poles to obtain a first signal, which may be processed by the processing module in order to detect a potential abnormality in the physiological signal. If an abnormality is detected, this may be interpreted as a change in a physiological state of the patient, and accordingly, for example, a message to an external device may be transmitted to trigger an alert in a home monitoring system.

In order to discern a true abnormality due to a change in a physiological condition of the heart from a false alert, herein, the first signal is assessed using a second signal received by another, second pair of electrode poles other than the first pair of electrode poles used to sense the first signal. If it is found that an indication of a potential abnormality is not detected in the second signal and hence cannot be verified based on the second signal, it may be assumed that the first signal is inconsistent, a signal change in the first signal being likely due to other factors, for example a signal loss due to a mechanical or electrical failure of the implantable medical device or a loss of coupling to surrounding tissue.

Hence, using the arrangement of multiple electrode poles, different signals may be received, wherein one signal may be assessed for consistency based on another signal and vice versa. Hence, by processing multiple signals a risk for a false alarm can be reduced, in that an abnormality in a signal can be verified based on another signal. Only if the second signal is found to be consistent with the first signal, an abnormality detected based on the first signal is assumed to be due to a change in a physiological state of the patient, such that an alarm, for example, in a home monitoring system may be triggered.

In one embodiment, the arrangement of multiple electrode poles, the arrangement of electrode poles or the arrangement of at least the first electrode pole, the second electrode pole and the third electrode pole (three different terms for the same arrangement) contains four or more electrode poles. Thus, the mentioned risk for a false alarm can be further reduced.

In one embodiment, the electrode poles are arranged aligned along a longitudinal axis.

In one embodiment, the first electrode pole and the second electrode pole define a first signal reception vector therebetween pointing along the longitudinal axis, the second electrode pole and the third electrode pole define a second signal reception vector therebetween pointing along the longitudinal axis, and the third electrode pole and the first electrode pole define a third signal reception vector therebetween pointing along the longitudinal axis. Because the electrode poles are aligned along the longitudinal axis, the signal reception vectors are pointing in parallel to one another, but due to the different axial locations of the different electrode poles differ from one another. One pair of electrode poles, for example the first electrode pole and the second electrode pole, hence senses a different signal than another pair of electrode poles, for example the first electrode pole and the third electrode pole. If, for example, a mechanical or electrical failure or a loss of coupling occurs at the second electrode pole, a signal sensing at the pair of the first electrode pole and the third electrode pole is not impacted. A signal received by one pair of electrode poles hence may be checked for consistency using another signal received by another pair of electrode poles, hence allowing to reduce a risk for a false alert in case, e.g., an asystole or cardiac fibrillation is identified in an electrocardiogram signal sensed by a particular pair of electrode poles.

In one embodiment, the implantable medical device comprises a housing, wherein the first electrode pole is arranged at a first end of the housing, the second electrode pole is arranged at a second end of the housing, and the third electrode pole and/or any further electrode pole is arranged at a location in between said first end and said second end. The housing may, for example, have a rigid shape, wherein the electrode poles may be formed by certain housing segments, or may be formed by discrete pole elements arranged on the housing. For example, the housing may comprise a first housing segment in which the processing module is received, a second housing segment in which a battery module is received, and a third housing segment, for example, formed by a so-called header portion extending, for example longitudinally, from another housing segment.

In particular in case the first electrode pole is arranged on a first end of the housing and the second electrode pole is arranged at a second end of the housing opposite to the first end, the first pair of electrode poles used to sense said first signal may beneficially be formed by the first electrode pole and the second electrode pole. The first electrode pole and the second electrode pole in this case may form a comparatively long signal reception vector, hence allowing for a reception of comparatively strong physiological signals from regions remote to the implantable medical device. In this case, the second pair of electrode poles may be formed by the first and the third/further or the second and the third/further electrode pole, defining a signal reception vector which is shorter than the first signal reception vector in between the first electrode pole and the second electrode pole.

In one embodiment, the processing module is configured to process different signals received by different pairs of electrode poles in different processing channels. In particular, the processing module may be configured to process the first signal received by the first pair of electrode poles in a first processing channel and the second signal received by the second pair of electrode poles in a second processing channel. Processing of the different signals hence may take place in different channels, wherein within each channel, for example, an amplification, a filtering and/or an analog-to-digital conversion may take place.

The processing of the different signals herein beneficially takes place synchronously, such that the first signal received via the first pair of electrode poles is processed synchronously with the second signal received via the second pair of electrode poles. Hence, the first signal may be assessed for consistency based on the second signal in that the first signal may be compared or in another way evaluated based on the second signal, and only if a finding in the first signal is verified according to the second signal, the finding is assumed to be a true finding.

Hence, using the arrangement of multiple (more than two) electrode poles different signals are synchronously received and processed. Based on the different signals a verification of one signal using another signal may take place.

In one embodiment, the processing module is configured to assess the consistency of the first signal based on a comparison of the first signal and the second signal. For example, if an asystole is detected in the first signal, it may be assessed by comparing the first signal to the second signal whether an asystole likewise is also detected in the second signal. Only if this is the case it is assumed that a true asystole is present, and accordingly an alarm may be triggered, for example, in a home monitoring system.

In one embodiment, the processing module is configured to assess the consistency of the first signal by feeding the first signal and the second signal to a combiner and by processing a combined signal output by the combiner. Using the combiner, for example, a signal summation (signal A+signal B), a signal difference (signal A-signal B) or a signal relation (signal A/signal B) may be formed. Based on the combined signal it may be assessed whether a change in the first signal is due to a change in a physiological condition or due to another factor.

In one embodiment, the processing module is configured, for assessing the consistency of the first signal, to evaluate said second signal for detection of at least one cardiac event in the second signal. For example, using the second signal a sequence of ventricular events (Vs) and/or atrial events (As) may be determined, and based on the cardiac events, for example, a heart rate may be derived. If an asystole is identified in the first signal, but a regular heart rate is identified in the second signal, the asystole finding can be assumed to be due to an inconsistency in the first signal.

In one embodiment, the processing module is configured to assess the consistency of the first signal in case a signals loss is detected in the first signal or in case an asystole is detected in the first signal. The first signal hence is not at all times checked for consistency, but only in case an abnormality is identified in the first signal. This may allow to reduce processing effort, in that a verification of the first signal is carried out only in case of a particular finding in the first signal.

In one embodiment, the processing module is configured, in case an inconsistency in the first signal is identified, to monitor cardiac activity based on another signal received by a pair of electrode poles of the arrangement of at least the first electrode pole, the second electrode pole and the third electrode pole other than the first pair. Hence, if a signal received using the first pair of electrode poles is found to be inconsistent with a second signal, the processing module may switch to another pair of electrode poles to continue sensing, such that sensing may be reliably continued in case, for example, a signal loss due to a mechanical or electrical failure or a loss of coupling occurs at the first pair of electrode poles. The processing module hence may switch adaptively between the different pairs of electrode poles defined by the arrangement of electrode poles, such that a sensing may take place by one pair of electrode poles if a sensing cannot be reliably conducted using another pair of electrode poles.

If the implantable medical device is used in a home monitoring system, the implantable medical device may comprise communication circuitry configured to establish a communication to an external device which is external to the patient. In case an inconsistency in the first signal is identified, the processing module may, for example, generate and transmit a warning message to the external device, such that the external device is informed about a potential failure at the implantable medical device.

In another aspect, a method for operating an implantable medical device for sensing physiological signals, for example electrocardiogram signals, comprises: sensing physiological signals using an arrangement of at least a first electrode pole, a second electrode pole and a third electrode pole: processing, using a processing module, signals received via said arrangement of at least the first electrode pole, the second electrode pole and the third electrode pole: monitoring, using the processing module, cardiac activity based on a first signal received by a first pair of electrode poles of the arrangement of at least the first electrode pole, the second electrode and the third electrode pole; and assessing, using the processing module, a consistency of said first signal based on a second signal received by a second pair of electrode poles of the arrangement of at least the first electrode pole, the second electrode and the third electrode pole different then said first pair.

The advantages and advantageous embodiments described above for the implantable medical device equally apply to the method, such that it shall be referred to the above in this respect.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The various features and advantages of the present invention may be more readily understood with reference to the following detailed description and the embodiments shown in the drawings. Herein,

FIG. 1 shows a schematic drawing of an implantable medical device implanted in a patient;

FIG. 2 shows a schematic drawing of an embodiment of an implantable medical device comprising an arrangement of electrode poles;

FIG. 3 shows a schematic drawing of another embodiment of an implantable medical device;

FIG. 4 shows a schematic drawing of an implantable medical device, illustrating a signal reception using a pair of electrode poles;

FIG. 5 shows a drawing of an implantable medical device, illustrating a signal reception using different signal vectors formed by different pairs of electrode poles;

FIG. 6 shows a schematic drawing of a processing of received signals; and

FIG. 7 shows a schematic drawing of another embodiment of a processing of received signals.

### DETAILED DESCRIPTION

Subsequently, embodiments of the present invention shall be described in detail with reference to the drawings. In the drawings, like reference numerals designate like structural elements.

It is to be noted that the embodiments are not limiting for the present invention, but merely represent illustrative examples.

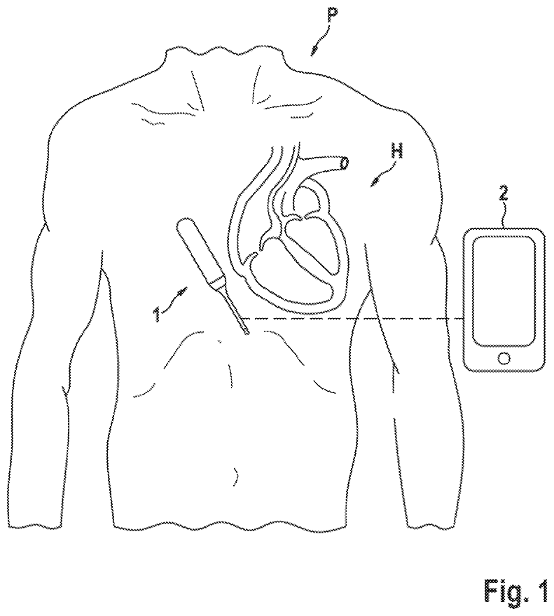

Referring to FIG. 1, in one embodiment an implantable medical device 1 is implanted (for example, subcutaneously) into a patient P for serving a therapeutic and/or diagnostic function. The implantable medical device 1 may, for example, be implanted subcutaneously into the patient P for monitoring cardiac activity of the patient's heart H. The implantable medical device 1, for this, comprises an arrangement of electrode poles which are used to couple to surrounding tissue and to sense physiological signals, here electrocardiogram signals, originating from the heart H.

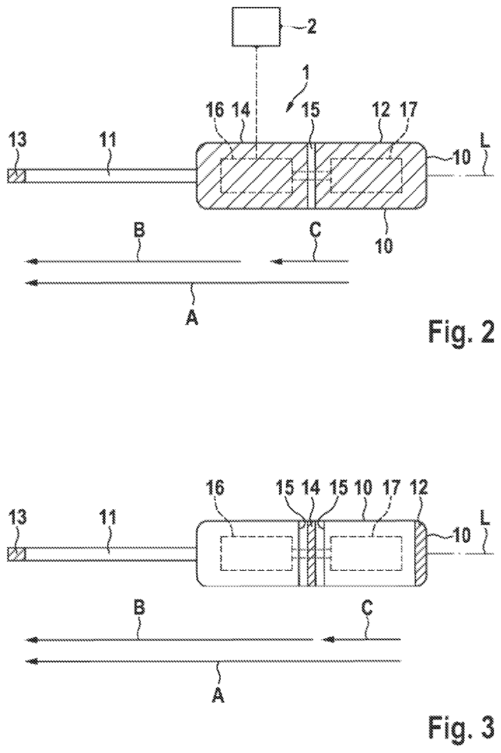

Referring now to FIG. 2, in one embodiment the implantable medical device 1 comprises a housing 10 formed by different housing segments, the housing 10 enclosing and encapsulating a processing module 16 formed by electronic circuitry and a battery module 17. In particular, a first housing segment may receive and enclose the processing module 16, whereas a second housing segment receives and encloses the battery module 17. Another housing segment 11 longitudinally extends from the first and second housing segments and forms a header portion having reduced cross-sectional dimensions with respect to the other housing segments.

In the embodiment of FIG. 2, a first electrode pole 12 is formed by the housing segment enclosing the battery module 17, a second electrode pole 13 is arranged at a far end of the housing segment 11 forming the header portion, and a third electrode pole 14 is formed by the housing segment enclosing the processing module 16. The implantable medical device 1 with its housing 10 generally extends along a longitudinal axis L, the electrode poles 12, 13, 14 being aligned along the longitudinal axis L and being axially displaced with respect to one another along the longitudinal axis L. The electrode poles 12, 13, 14 herein are electrically separated from one another, an electrically insulating segment 15 being arranged in between the electrode poles 12, 14 formed on the main housing portion and the header portion formed by the housing segment 11 separating the electrode pole 13 from the other two electrode poles 12, 14.

At this point it should be explicitly pointed out that an alignment of the electrode poles 12, 13, 14, any housing segments or other elements along the longitudinal axis L is not absolutely necessary. Other arrangements are also possible. For example, the electrode poles 12, 13, 14 may be arranged alternately on a top and a bottom, or on a left and a right side of the implantable medical device.

In the embodiment of FIG. 2, the electrode poles 12, 13, 14 may be formed by portions of the housing 10 itself, the housing 10 being made, for example, from an electrically conductive material, in particular a metal material. By exposing portions of the housing 10 towards the outside, the electrode poles 12, 13, 14 are formed and may electrically contact with surrounding tissue in order to establish a coupling between the electrode poles 12, 13, 14 to the surrounding tissue.

Referring now to FIG. 3, in another embodiment the first electrode pole 12 is formed at an end of a housing segment of the housing 10 encapsulating the battery module 17, whereas the electrode pole 14 is formed by an electrode element which is electrically insulated from other portions of the housing 10 by the electrically insulating segments 15. For example, a multilayered pole element may be employed for forming the electrode pole 14, as it is described, for example, in EP 3 278 836 B1. The electrode pole 13 again is formed at a far end of the housing segment 11 forming the header portion.

In any of the embodiments of FIGS. 2 and 3, using the arrangement of electrode poles 12, 13, 14, electrocardiogram signals or other physiological signals may be received and processed by the processing module 16. Based on the processing, a communication with an external device 2 may be established, for example to transmit alert messages to the external device 2, for example, within the context of a home monitoring system for monitoring a physiological state of the patient P.

The different electrode poles 12, 13, 14 herein define signal reception vectors A, B, C by means of which signals may be received using pairs of associated electrode poles 12, 13, 14. In particular, a first signal reception vector A is formed between the first electrode pole 12 and the second electrode pole 13, a second signal reception vector B is formed between the third electrode pole 14 and the first second electrode pole 13, and a third signal reception vector C is formed between the first electrode pole 12 and the third electrode pole 14. As the first electrode pole 12 and the second electrode pole 13 are arranged at opposite ends of the housing 10, the associated signal reception vector A is longer than the other two signal reception vectors B, C.

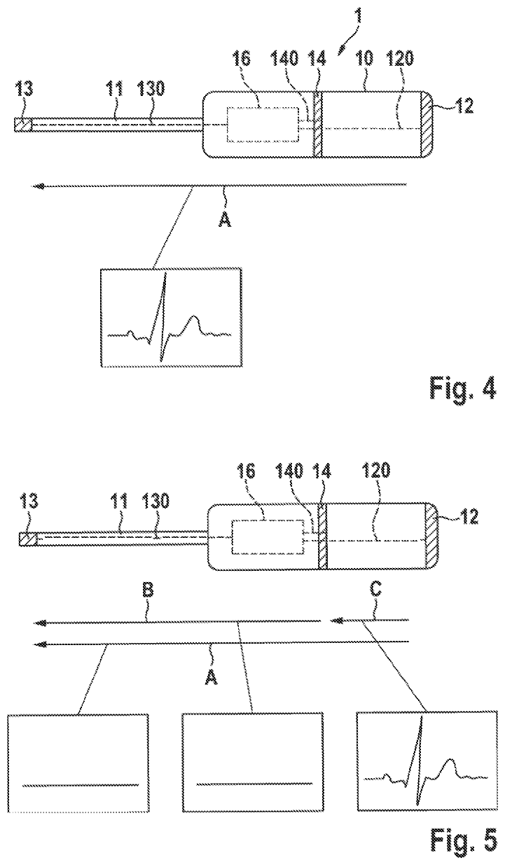

Referring now to FIG. 4, for a regular signal reception, for example, a pair of electrode poles formed by the first electrode pole 12 and the second electrode pole 13 may be used, defining a signal reception vector A as illustrated in FIG. 4. Using the signal reception vector A electrocardiogram signals or other physiological signals may be recorded and may be processed within the processing module 16, which is connected to the different electrode poles 12, 13, 14 by means of electrical connection lines 120, 130, 140. Using the signal reception vector A for regular signal reception may come with the benefit of a strong signal reception even from remote regions, as the electrode poles 12, 13 define a comparatively long signal reception vector A for receiving signals from surrounding tissue.

Generally, the processing module 16 may process a received signal in order to detect an abnormality within the electrocardiogram signal, in particular an asystole or a ventricular fibrillation. In case an asystole is identified, a message to the external device 2 may be generated and transmitted, such that an alert is triggered within an associated home monitoring system for alerting medical personnel to attend to the patient P.

As there is a general desire to reduce a risk of a false alarm in order to avoid a false triggering of an alert, an abnormality in a received signal due to a true change in a physiological state must be discerned from a signal change which is due to other factors, such as a mechanical or electrical failure of the implantable medical device 1 or a loss of coupling of one of the electrode poles 12, 13, 14 to surrounding tissue.

Referring now to FIG. 5, if the signal reception vector A defined by the electrode poles 12, 13 is used for a signal reception, a loss of coupling at the electrode pole 13 may give rise to a signal loss, as illustrated in FIG. 5, which may be mistaken for an asystole. Likewise, if the electrical line 130 connecting the electrode pole 13 to the processing module 16 breaks, a signal received via the signal reception vector A will be zero, which likewise may be mistaken for an asystole.

It hence is proposed herein to conduct, within the processing module 16, a consistency check in which a signal received by a pair of electrode poles 12, 13, 14 is assessed for consistency using another signal received by another pair of electrode poles 12, 13, 14.

In the example of FIG. 5, the signal received via the signal reception vector A may be assessed by comparison, e.g., to a signal received via the signal reception vector C defined in between the first electrode pole 12 and the third electrode pole 14, which is not impacted by a failure or loss of coupling at the electric pole 13. Hence, by comparing the signal received via the signal reception vector C with the signal received via the signal reception vector A it can be identified that a flat, zero signal at the signal reception vector A likely is not due to a change in physiological condition, but due to another factor. An asystole identified based on the signal reception vector A hence can be interpreted as a false alarm, as the signal received via the signal reception vector C indicates that in fact no asystole is present.

Hence, when using a particular pair of electrode poles 12, 13, 14, for example the pair formed by the first electrode pole 12 and the second electrode pole 13, for regular signal reception, the other two pairs of electrode poles 12, 14:13, 14 can be used for assessing the consistency of the signal reception using the first pair of electrode poles 12, 13. Based on a comparison of the signals received by the different signal reception vectors A, B, C it can be assessed whether an abnormality in one signal reception vector is due to a physiological condition or due to another factor, such as a mechanical or electrical failure of the implantable medical device 1 or a loss of coupling.

For the processing, in principle different approaches can be chosen.

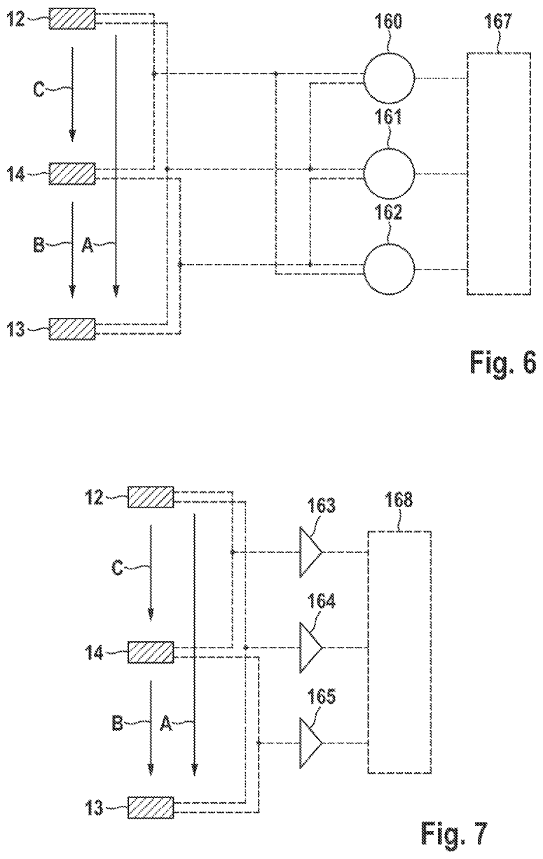

For example, as shown in FIG. 6, signals received via the different signal reception vectors A, B, C may be fed to combiners 160, 161, 162, the combiners 160, 161, 162 forming combined signals and forwarding the combined signals to a processing circuitry 167 for a further processing. For example, in the combiners 160, 161, 162 a summation of the signals (e.g., signal A+signal B), a difference of the signals (e.g., signal A-signal B), or a relation of the signals (e.g., signal A/signal B) may be formed and may be processed.

In another example, shown in FIG. 7, the different signals sensed via the different signal reception vectors A, B, C may each be fed to an amplifier 163, 164, 165 and to a processing circuitry 168, which, for example, may digitize the signals and may process signals in the digital domain.

Generally, the signals may be processed in different signal channels, the signals being received synchronously using the different signal reception vectors A, B, C, such that a synchronous assessment of one signal received via one pair of electrode poles 12, 13, 14 with respect to a signal received via another pair of electrode poles 12, 13, 14 is enabled. Within the processing, a comparison may take place, for example by comparing signal levels with respect to one another. In another embodiment, waveforms may be compared, for example a timing or level of a QRS waveform, a P wave or a T wave.

Generally, a processing may take place in one or multiple channels. If, for example, only one processing channel involving a single signal amplification is used, a signal multiplexing may be employed. The processing may include the use of one or multiple comparators.

In another embodiment, cardiac events may be compared. For example, if an asystole is identified in the first signal reception vector A being defined between the first electrode pole 12 and the second electrode pole 13, cardiac events such as ventricular contraction events (Vs) and/or atrial contraction events (As) may be identified in another signal, for example obtained via the signal reception vector C between the first electrode pole 12 and the third electrode pole 14, as indicated in FIG. 5, wherein based on cardiac events in the other signal, for example, a heart rate may be identified. Based on the detected cardiac events of the second signal an asystole in the first signal may be found to be inconsistent, such that no alarm based on the asystole detected in the first signal is raised.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Implantable medical device
10 Housing
11 Housing segment
12 Electrode pole
120 Connection line
13 Electrode pole
130 Connection line
14 Electrode pole
140 Connection line
15 Electrically insulating segment
16 Processing module
160-162 Combiner
163-165 Amplifier
167, 168 Processing circuitry
17 Battery module
2 External device
A, B, C Signal reception vector
H Heart
L Longitudinal axis
P Patient

1. An implantable medical device for sensing physiological signals, comprising:
an arrangement of at least a first electrode pole, a second electrode pole and a third electrode pole, said arrangement of at least the first electrode pole, the second electrode and the third electrode pole being configured to sense physiological signals; and
a processing module for processing signals received via said arrangement of at least the first electrode pole, the second electrode pole and the third electrode pole;
wherein the processing module is configured to monitor cardiac activity based on a first signal received by a first pair of electrode poles of the arrangement of at least the first electrode pole, the second electrode and the third electrode pole and to assess a consistency of said first signal based on a second signal received by a second pair of electrode poles of the arrangement of at least the first electrode pole, the second electrode and the third electrode pole different then said first pair.

2. The implantable medical device according to claim 1, wherein the arrangement contains four or more electrode poles.

3. The implantable medical device according to claim, wherein the electrode poles are arranged aligned along a longitudinal axis.

4. The implantable medical device according to claim 3, wherein the first electrode pole and the second electrode pole define a first signal reception vector therebetween pointing along the longitudinal axis, the second electrode pole and the third electrode pole define a second signal reception vector therebetween pointing along the longitudinal axis, and the first electrode pole and the third electrode pole define a third signal reception vector therebetween pointing along the longitudinal axis.

5. The implantable medical device according to claim 1, comprising a housing, wherein the first electrode pole is arranged at a first end of the housing, the second electrode pole is arranged at a second end of the housing opposite the first end, and the third electrode pole and/or any further electrode pole is arranged at a location in between said first end and said second end.

6. The implantable medical device according to claim 1, wherein said first pair of electrode poles is formed by the first electrode pole and the second electrode pole.

7. The implantable medical device according to claim 1, wherein the processing module is configured to process said first signal in a first processing channel and said second signal in a second processing channel.

8. The implantable medical device according to claim 7, wherein the processing module is configured to perform, in said first processing channel and/or in said second processing channel, at least one of an amplification and an analog-to-digital conversion.

9. The implantable medical device according to claim 7, wherein the processing module is configured to synchronously process said first signal in said first processing channel and said second signal in said second processing channel.

10. The implantable medical device according to claim 1, wherein the processing module is configured to assess said consistency of the first signal based on a comparison of the first signal and the second signal.

11. The implantable medical device according to claim 1, wherein the processing module is configured, for assessing said consistency of the first signal, to assess at least one of a signal summation of said first signal and said second signal, a signal difference between said first signal and said second signal, and a signal relation of said first signal and said second signal.

12. The implantable medical device according to claim 1, wherein the processing module is configured, for assessing said consistency of the first signal, to evaluate said second signal for detection of at least one cardiac event in the second signal.

13. The implantable medical device according to claim 1, wherein the processing module is configured to assess said consistency of the first signal in case a signal loss is detected in said first signal or in case an asystole or a cardiac fibrillation is detected in said first signal.

14. The implantable medical device according to claim 1, wherein the processing module is configured, in case an inconsistency in said first signal is identified, to monitor cardiac activity based on another signal received by a pair of electrode poles of the arrangement of the first electrode pole, the second electrode and the third electrode pole other than said first pair.

15. A method for operating an implantable medical device for sensing physiological signals, comprising:
sensing physiological signals using an arrangement of at least a first electrode pole, a second electrode pole and a third electrode pole;
processing, using a processing module, signals received via said arrangement of at least the first electrode pole, the second electrode pole and the third electrode pole;
monitoring, using the processing module, cardiac activity based on a first signal received by a first pair of electrode poles of the arrangement of at least the first electrode pole, the second electrode and the third electrode pole; and
assessing, using the processing module, a consistency of said first signal based on a second signal received by a second pair of electrode poles of the arrangement of at least the first electrode pole, the second electrode and the third electrode pole different then said first pair.
